(19) United States
(12) Patent Application Publication
(10) Pub. No.: US 2016/0143857 A1
(43) Pub. Date: May 26, 2016

(54) HYBRID ALGINATE-SILICA BEADS AND METHOD FOR OBTAINING THEM

(71) Applicant: UNIVERSITE DE NAMUR, Namur (BE)

(72) Inventors: Jonathan DESMET, Champion (BE); Christophe MEUNIER, Floreffe (BE); Bao-Lian SU, Saint-Servais (BE)

(21) Appl. No.: 14/900,486

(22) PCT Filed: Jun. 17, 2014
(86) PCT No.: PCT/EP2014/062765

§ 371 Date: Dec. 21, 2015

(30) Foreign Application Priority Data
Jun. 27, 2013 (EP) 13174004.5

(51) Int. Cl. A61K 9/50; A61K 36/05

(52) U.S. Cl. CPC A61K 9/5036; A61K 36/05; A61K 9/5089; A61K 9/501

(57) ABSTRACT

A hybrid silica bead having a millimeter scaled-size is adapted for the entrapment of a component or a bioactive substance. The bead is formed of a porous core including a hybrid alginate-silica and an external porous layer having silica and a silica concentrator. A one-pot process prepares hybrid beads for use of the beads for the entrapment of a component or a bioactive substance.

### FIELD OF THE INVENTION

The present invention relates to hybrid alginate-silica beads and to a one-pot process for the preparation of these hybrid beads.

The present invention is also related to the use of the beads according to the invention. Beads of the invention are used for the entrapment of biologically active entities in a broad range of fields for example in bioreactors, biocatalysts, biosensors, chromatographic columns, etc. Particularly, the new beads according to the invention are used for the entrapment of enzymes, organelles such as thylakoids, vacuoles, chloroplasts, vesicles or for the entrapment of whole cells such as microalgae, bacteria, yeast, animal or plant cells. Such entrapments aim at producing high value metabolites, such as carotenoids, hormones, proteins, (processed) pro-drugs or a mixture thereof.

### STATE OF THE ART

Calcium alginate capsules can be easily synthesized by extruding a sodium alginate solution into an aqueous solution of calcium chloride and enable to maintain the biological activity of entrapped living microorganisms. However, these calcium alginate capsules show poor mechanical stability. It is known that alginate is a swelling component which leads over time to leakage of entrapped components, including living cells which can subsequently be released and maybe proliferate in the external medium. Indeed, fractures are observed on the entire bead volume and the strength of the capsule decreases from the surface to the core. Therefore, alginate capsules would seem not to be the appropriate host matrix for the encapsulation of components including living cells.

The synthesis of hybrid alginate-silica capsules exhibiting limited mechanical resistance has already been reported. The most common approaches involve either a multi-step process or a layer-by-layer process. The synthesis of hybrid alginate-silica mineralized beads through a two-step process has been disclosed by Dandoy et al. (P. Dandoy, C. F. Meunier, C. Michiels, B.-L. Su, Plos One, 2011, 6, 1-12). The obtained beads composed of two layers, an alginate-silica composite core and a Ca-alginate layer, are used for entrapping active mammalian cells. However, the dissolution of silica is a phenomenon which occurs over time and leads to the release of the beads' content in the external medium.

Patent application FR 2842438 A1 discloses a process for preparing beads containing a cross-linked mineral matrix. The process is suitable for the preparation of alumina- or silica-based millimeter-scale beads by a sol-gel process. The production of these beads comprises the step of preparing gelled beads by pouring a suspension comprising a precursor of the inorganic matrix and an alginate dropwise into a solution of a polyvalent cation salt, at a pH of less than 3. The combined actions of the polyvalent cation and of the acidity variations of the medium contribute to the gelling of this alginate and to a congealing of the drops as “soft” beads. Thus, the mineral matrix is homogeneously distributed throughout the bead. However, dissolution of silica occurs over time in these prepared hybrid alginate-silica beads as observed by Dandoy et al (2011).

Chen et al. (Process biochemistry vol 42, No. 6, pp. 934-942, 2007) discloses alginate-silicate beads including Pseudomonus Puteola cells for decolorization of Azo dye (reactive Red 22). These beads were made of a dense silicate gel layer coating a macroporous alginate-silicate core having improved mechanical stability.

Coradin et al. (Applied microbiology and biotechnology, Vol. 61, No. 5-6, pp. 429-434, 2003) discloses that the optimization of membrane properties of silica-alginate composite microcapsules exhibiting may enhances their mechanical, thermal and diffusion properties.

U.S. Pat. No. 4,797,358 discloses a microorganism or enzyme immobilization with a mixture of alginate and silica sol. This mixture is contacted with a gelling agent in the form of an aqueous solution to obtain a gel containing this microorganism or enzyme.

Lu et al (Catalysis today, Vol. 115, No. 1-4, pp. 263-268, 2006) discloses an enzyme encapsulated in an alginate-silica hybrid gel and alginate silica gel beads.

### AIMS OF THE INVENTION

A main aim of the invention is to provide new hybrid alginate-silica beads and a method for obtaining them, neither of which presents the drawbacks of the state of the art.

In particular, the present invention aims to provide new, preferably transparent and preferably spherical beads, as well a simple eco-friendly and efficient one-pot method for obtaining them, these beads exhibiting good mechanical and chemical stability characteristics and in which the dissolution rate of silica species is reduced over time or is prevented.

A further aim of the present invention is to provide such beads that can be used in various fields, especially for the entrapment of components or bioactive substances, such as enzymes, cell organelles, such as thylakoids, vacuoles, chloroplasts, vesicles, but also whole cells such as microalgae, bacteria, yeast, plant or animal cells.

### Main Technical Features of the Invention

The present invention relates to (hybrid silica) beads having a millimeter-scale size adapted for the entrapment of (and preferably comprising) components or bioactive substances, wherein the beads comprise a porous core and a porous shell, the porous core comprising a hybrid alginate-silica and the external porous shell comprising silica and a silica concentrator (such as a polycationic organic polymer).

Preferably, the diameter of the millimeter-scale size ranges from (about) 0.5 mm to (about) 5 mm. The thickness of the porous shell is preferably comprised between (about) 1 μm and (about) 10 μm. The shell comprises pores having a size ranging from (about) 1 nm to (about) 500 nm.

In the present invention, alginate (alginic acid) is defined as an anionic polysaccharide distributed widely in the cell walls of brown algae. Alginate is a linear copolymer with homopolymeric blocks of (1-4)-linked β-D-mannuronate (M) and its C-5 epimer α-L-glucuronate (G) residues, respectively, covalently linked together in different sequences or blocks. The chemical compound sodium alginate is the sodium salt of alginate. Its empirical formula is NaC₆H₇O₆. Sodium alginate is a gum, extracted from the cell walls of brown algae.

(Hybrid) Beads according to the invention are advantageously prepared through a coacervation process which relies on the decrease in solubility of the hybrid sol containing one or more silica precursor(s) and an alginate solution, due to the addition of a silica concentrator (such as a polycationic organic polymer). In fact, the alginate acts as a template and the silica concentrator plays both the role of a concentrator of silicate and that of a catalyst to accelerate the hydrolysis and polycondensation of silica precursor(s) at the periphery of the bead, thus creating a porous crust (shell). The core of the beads is composed of a sodium alginate-silica composite in which components or bioactive substances, such as enzymes, organelles such as thylakoids, vacuoles, chloroplasts, vesicles, or living cells are encapsulated (entrapped). The obtained external layer (shell) of the bead is formed of a porous layer of silica concentrated by the silica concentrator.

According to preferred embodiments, the hybrid silica beads are further limited by one or more of the following technical features:
the silica precursor(s) used to prepare the beads is (are) selected from the group consisting of a polysilicic acid (H₂SiO₃)ₙ (preferably metasilicic acid H₂SiO₃), ormosils (organic modified silicas), a silica hydroxide, a silica alkoxide (such as tetramethyl orthosilicate (TMOS), tetraethyl orthosilicate (TEOS), tetrapropyl orthosilicate (TPOS), tetrakis(2-hydroxyethyl) orthosilicate (EGMS), tetrakis(2-hydroxypropyl) orthosilicate (PGMS) and tetrakis(2,3-dihydroxypropyl) orthosilicate (GLMS)), a silicate (such as sodium (Na₂SiO₃) or potassium silicate), silica nanoparticules, sorbitylsilane, trimethoxymethylsilane, dimethoxydimethylsilane, TMOS (tetramethoxysilane) DGS (diglycerylsilane), or a mixture thereof;
the silica concentrator is a polycationic organic polymer, preferably a long chain polyamine, preferably selected from the group consisting of polycation poly(diallyldimethylammonium) chloride (PDADMAC); spermine; cholesteryl spermine; spermidine; spermidine tryhydrochloride; spermidine phosphate hexahydrate; L-arginyl-3,4-spermidine; 1-4-butanediamine N-(3-aminopropyl)-monohydrochloride; putrescine (1,4-diamino-butane); 1,3-diamino-propane; 1,7-diamino-heptane; 1,8-diamino-octane; poly(allylamine) hydrochloride; poly(ethyleneimine); poly(N-methylethyleneimine); poly(N-vinyl-2-pyrrolidone); poly(2-(dimethyl-amino)ethyl methacrylate; chitosan; poly(vinylamine) hydrochloride; poly(propyleneimine); poly(N-methylpropyleneimine); poly(acrylamide-co-diallyldimethylammonium) chloride; poly-L-lysine; poly-L-arginine andpoly-L-histidine or a mixture thereof
the preferred long chain polyamine is PDADMAC;
the alginate used in the beads' formation may be an alginate of an alkaline metal, preferably sodium alginate;
the (external) porous shell comprises pores having a size ranging from (about) 1 to (about) 500 nm;
the thickness of the (external) porous shell is comprised between (about) 1 and (about) 10 μm;
the content in silica of the (external) porous shell is comprised between (about) 0.1 and (about) 1 M, preferably between (about) 0.5 and (about) 0.8 M;
an intermediate layer of hybrid calcium alginate-silica is formed between the porous core and the (external) porous shell;
the components or bioactive substances entrapped or encapsulated in the bead(s) are preferably biological or organic substances having a bioactive effect (such as a therapeutic, neutraceutic, cosmetic or biochemical (anabolic or catabolic) activity) upon a cell, tissue, organ or biological substrate (preferably a plant or animal, more preferably a mammal (including a human) cell, tissue or organ) or being a cell, preferably this bioactive substance is selected from the group consisting of an enzyme, a (monoclonal) antibody an antigenic binding portion of a (monoclonal) antibody, an hormone, a vitamin, an active drug (i.e. insulin) or prodrug or a whole cell, such as microalgae, bacteria, fungi including yeast, plant or animal cells or an organelle of a cell, preferably a photosynthetically active cell (microalgae, or plant cells) or a mixture thereof.

The invention also relates to a one-pot method for the preparation of (hybrid silica) beads according to the invention, which comprises the steps of:
mixing one or more silica precursor with a solution of alginate, the pH of the solution being comprised between (about) 2 and (about) 10, preferably between (about) 4 and (about) 6, and more preferably (about) 5, and with one or more component or bioactive substance as above defined (preferably a cell, such as microalgae) to be encapsulated (or entrapped) in said beads;
dropping the mixture into an aqueous solution of a silica concentrator;
incubating the obtained beads for a period comprised between (about) 1 minute and (about) 24 hours, preferably between (about) 30 minutes and (about) 180 minutes, more preferably about 3 hours; and
possibly transferring the obtained beads into an appropriate culture medium.

The method of the invention is carried out at a temperature of between (about) 10° C. and (about) 60° C., preferably at room temperature.

Advantageously, the method of the invention is further limited by one or more of the following technical features:
the aqueous solution of the silica concentrator further comprises a cationic salt (such as CaCl₂). The use of cationic salts improves the optical transparency of the obtained beads and avoids their aggregation.
the silica precursor(s) used to prepare the beads is (are) preferably a polysilicic acid (H₂SiO₃)ₙ (preferably metasilicic acid H₂SiO₃), ormosils (organic modified silicas), a silica hydroxide, a silica alkoxide (such as tetramethyl orthosilicate (TMOS), tetraethyl orthosilicate (TEOS), tetrapropyl orthosilicate (TPOS), tetrakis(2-hydroxyethyl) orthosilicate (EGMS), tetrakis(2-hydroxypropyl) orthosilicate (PGMS) and tetrakis(2,3-dihydroxypropyl) orthosilicate (GLMS)), a silicate (such as sodium (Na₂SiO₃) or potassium silicate), silica nanoparticules, sorbitylsilane, trimethoxymethylsilane, dimethoxydimethylsilane, TMOS (tetramethoxysilane), DGS (diglycerylsilane), or a mixture thereof. More preferably, the silica precursor is the polysilicic acid (H₂SiO₃)ₙ, trimethoxymethylsilane, dimethoxydimethylsilane or a mixture thereof.
the silica concentrator is a polycationic organic polymer, preferably a long chain polyamine, preferably selected from the group consisting of polycation poly(diallyldimethylammonium) chloride (PDADMAC); spermine; cholesteryl spermine; spermidine; spermidine tryhydrochloride; spermidine phosphate hexahydrate; L-arginyl-3,4-spermidine; 1-4-butanediamine N-(3-aminopropyl)-monohydrochloride; putrescine (1,4-diamino-butane); 1,3-diamino-propane; 1,7-diamino-heptane; 1,8-diamino-octane; poly(allylamine) hydrochloride; poly(ethyleneimine); poly(N-methylethyleneimine); poly(N-vinyl-2-pyrrolidone); poly(2-(dimethyl-amino)ethyl methacrylate; chitosan; poly(vinylamine) hydrochloride; poly(propyleneimine); poly(N-methylpropyleneimine); poly(acrylamide-co-diallyldimethylammonium) chloride; poly-L-lysine; poly-L-arginine; poly-L-histidine or a mixture thereof.

Preferably, in the method according to any of the invention, the concentration of the silica precursor is comprised between (about) 0.1 M and (about) 2 M, the concentration of the alginate is preferably comprised between (about) 0.5% wt and (about) 5% wt and the concentration of the silica concentrator, preferably the silica concentration, preferably the polycation PDADMAC is comprised between (about) 0.4% wt and (about) 10% wt.

Chemical factors influencing the size of the pores on the (external) shell of the beads include but are not limited to the concentration of the silica precursor(s), the volume ratio between silica precursor(s) and the alginate solution, the percentage (in mass) of alginate, the incubation time in the coacervation solution, the percentage (in mass) of the polycationic organic polymer.

Several physical factors can modulate the beads' diameter, such as the diameter of the needle used to extrude the sol silica/alginate, the height at which the sol silica/alginate is dropped into the long chain polyamine solution, the speed at which the sol silica/alginate is dropped into the polycationic organic polymer solution, or the time of incubation of the beads in the coacervation solution.

Furthermore, several physical factors can modulate the beads' physical resistance, including, but not limited to the time of incubation of the beads in the coacervation solution, as longer incubation times increased the beads' Young Modulus.

Moreover, the mechanical resistance of the hybrid silica-alginate beads of the invention can be improved by adding additives, such as silica colloids (e.g., LUDOX®), silica co-precursors, or nanoparticles of silica to the silica precursor solution. Those additives function as additional sources of silica.

This simple (easy-handling and low cost technology), rapid, eco-friendly and efficient method is advantageous, because it is neither toxic for the environment nor for the entrapped cells which can be kept alive and divide for a long time (up to several months) in the beads. The beads carry a porous structure throughout their entire volume, allowing for an excellent diffusion of nutrients and metabolites to and from the cells within the beads.

This method allows the production of entrapped cells into transparent, robust and spherical beads that will improve the life span and biological activities of these cells and allow their use in numerous applications. Such applications include their incorporation into biosensors, biofuel cells or (photo)bioreactors for the production at high yields (e.g., green chemistry using CO₂ as reactant and light radiation as source of energy) of molecules of interest, such as pharmaceutical molecules (including (monoclonal) antibodies or portion(s) thereof (or similar products, such as nanobodies or alphabodies)), nutraceuticals or cosmetic molecules such as carotenoids (beta-carotene), vitamins, hormones or enzymes, all of which can easily be recovered from the external medium without requiring the killing of the cells.

These living cells entrapped into the beads can be also used for the delivery of active compounds (like insulin, a drug or a pro-drug, (monoclonal) antibodies or portion(s) thereof (or similar products, such as nanobodies or alphabodies)) into living organs of animals, including the human body.

The beads according to the invention having specific characteristics (controlled diameter and pore size) can also be used as such (without any entrapped elements or cells) in purification and/or separation devices and methods, for instance in chromatographic columns.

A last aspect of the present invention is related to the use of the beads according to the invention or the beads obtained by the method according to the invention in a bioreactor for the production of a molecule of interest, in delivery of a molecule of interest in a living organ of an animal including the humans and/or in purification and/or separation methods and devices, preferably in a chromatographic column.

### BRIEF DESCRIPTION OF THE FIGURES

FIG. 1 discloses the formation mechanism of (hybrid alginate-silica) beads of the invention. (1) represents a layer of hybrid sodium alginate-SiO₂, (2) PDADMAC, (3) represents a layer of hybrid calcium alginate-SiO₂.

FIG. 2 represents the photochemical production of oxygen by entrapped microalgae within hybrid alginate-silica beads according to the invention.

FIG. 3 represents the mechanical resistance of hybrid alginate-silica beads as compared to alginate capsules.

FIG. 4 represents the average diameter of hybrid alginate-silica beads as a function of the incubation time into a PDADMAC/CaCl₂ solution.

FIG. 5 represents the photochemical production of oxygen by entrapped microalgae within hybrid alginate-silica beads according to the incubation time into a PDADMAC/CaCl₂ solution. Measures were taken 0, 1, 4 and 7 days after entrapment.

### DESCRIPTION OF PREFERRED EMBODIMENTS OF THE INVENTION

The present invention will be described in more details in the following non-limiting examples with reference to the enclosed figures.

FIG. 1 presents the formation mechanism of (hybrid alginate-silica) beads of the invention. This formation relies on a coarcevation process in which the addition of a polycationic organic polymer (e.g., PDADMAC) decreases the solubility of a hybrid solution containing silica precursor(s) and sodium alginate. In fact, the alginate acts as a template and the PDADMAC plays the role of a silica concentrator. The core part of the beads contains a hybrid sodium alginate-silica 1, the intermediate layer 3 is composed of hybrid calcium alginate-silica and the external layer (shell) 2 comprises silica and the silica concentrator PDADMAC. The PDADMAC-containing layer reduces or prevents any leakage of silica species outside the beads.

### Example 1

### Photochemical Production of Oxygen by Entrapped Microalgae within Hybrid Alginate-Silica Beads

### Microalgae Cultivation

The strain of Dunaliella tertiolecta (ATCC-30929) liquid stock cultures were maintained in flasks at ambient temperature under fluorescent strip lighting and transferred into fresh medium culture once a month. ATCC 30929 was grown in sterile flasks filled with JOHNSONS medium culture.

### Hybrid Alginate-Silica Beads Entrapping Microalgae

The experimental procedure that was established to successfully synthesize hybrid alginate-silica beads through a one-pot process involves the preparation of a hybrid alginate-silica solution by mixing the polysilicic acid (H₂SiO₃) (5 mL, 0.1-2 M), adjusted at a pH between about 4 and about 6 with NaOH 0.1 M, with a solution of sodium alginate (5 mL, 0.5-5% wt.) and a living cell suspension of Dunaliella tertiolecta (ATCC-30929). Then, this mixture was dropped into an aqueous solution of polycation poly(diallyldimethylammonium) chloride (PDADMAC) (0.4-10% wt.) containing CaCl₂ (5-100 mM). After about 3 hours of incubation within this mixture, hybrid alginate-silica beads entrapping microalgae were washed three times with fresh medium culture prior to be transferred into sterile flask in presence of JOHNSONS culture medium.

When appropriate, the living cell suspension was omitted from the preparation and the hybrid alginate-silica beads were otherwise synthesized as described above.

### Photosynthetic Activity

The photosynthetic activity of hybrid beads containing microalgae was examined and monitored through oxygen production in a Clark's cell vessel purchased from HansaTech (Norfolk, England). The procedure implied putting in suspension of between 2 and 15 beads, preferably between 2 and 8 beads, preferably about three beads in 1 mL of JOHNSONS medium culture mixed with NaHCO₃ (10 μL, 0.6 M).

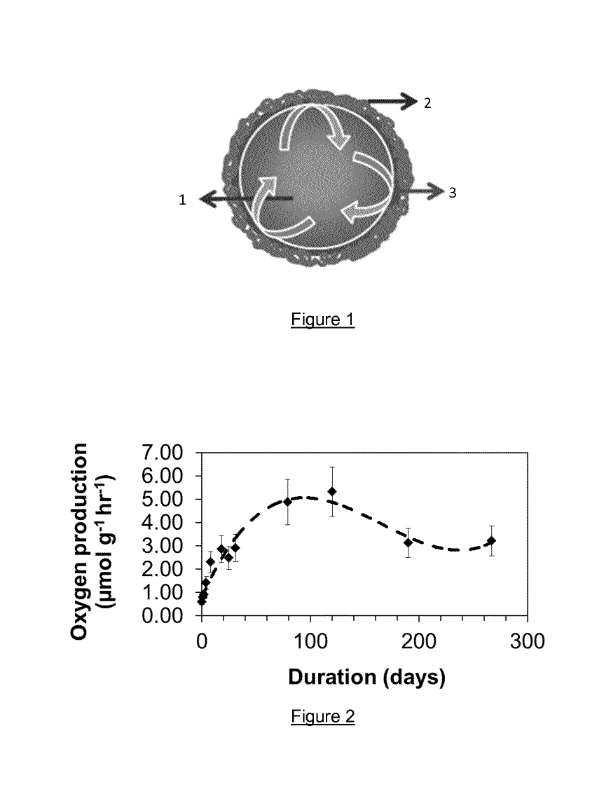

Microalgae entrapped within alginate-silica beads can produce oxygen for over 9 months as reported in FIG. 2. Time zero corresponds to the time when the microalgae were encapsulated within hybrid beads.

### Example 2

### Mechanical Resistance of Hybrid Alginate-Silica Beads as Compared to Alginate Capsules

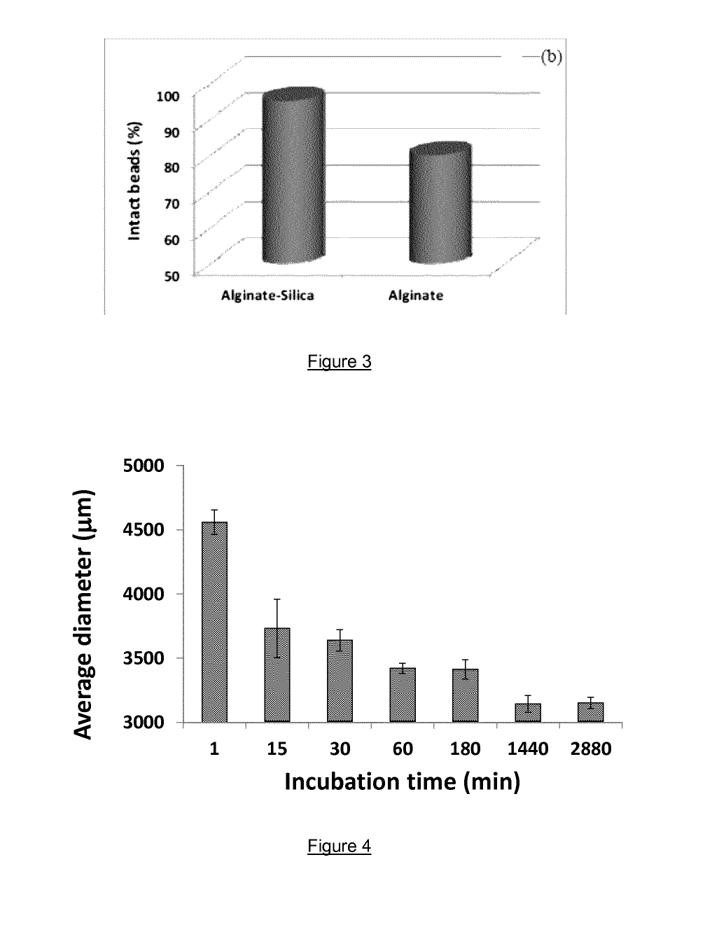

The experiment was performed as provided in example 1 with or without living cells. A comparative stability study of alginate and hybrid alginate-silica beads was realized. For this purpose, the beads were transferred into biological medium culture after synthesis. To evaluate their mechanical resistance, the beads were placed under stirring conditions at about 250 rpm for between about 1 hour and about 10 hours, preferably for about 2 hours within the medium culture and the beads were removed and the cracked beads counted. As shown in FIG. 3 and Table 1 herein below, alginate-silica beads exhibit a higher number of intact beads than the alginate beads. By increasing the incubation time into the PDADMAC/CaCl₂ solution, the mechanical resistance was also reinforced. The combination of silica with alginate thus reinforced the mechanical resistance of the hybrid beads.

TABLE 1
Mechanical resistance of beads of various compositions. The alginate/
PDADMAC and alginate/PDADMAC/silica beads were incubated for
about 1 hour in the PDADMAC/CaCl₂ solution. The alginate/silica
beads were incubated for about 1 hour in the CaCl₂ solution.
Their resistance was expressed as the value of their Young Modulus.
Values are given as means with standard deviations (n = 3).

| Composition of the beads | Young Modulus (E(kPa)) |
|---|---|
| Alginate/PDADMAC | 47 +/− 5 |
| Alginate/SiO2 | 90 +/− 20 |
| Alginate/SiO2/PDADMAC | 160 +/− 20 |

### Example 3

### Study of the Effect of the Incubation Time on the Average Diameter of Hybrid Alginate-Silica Beads

The experiment was performed as provided in example 1. The incubation time into the PDADMAC/CaCl₂ solution varied from 1 minute to 48 hours (2880 minutes). Additionally, a phenomenon of shrinkage of the beads was also observed over time, the latter can be explained by the polymerization process of silica within the PDADMAC/CaCl₂ solution which is more efficient over time and thus leads to a smaller size bead. The kinetic of the beads' shrinkage was graphically reported in FIG. 4. It appeared that the shrinkage is well pronounced during the first hours to reach a stable size after 24 hours (1440 minutes).

### Example 4

### Photochemical Production of Oxygen by Entrapped Microalgae within Hybrid Alginate-Silica Beads According to Incubation Time

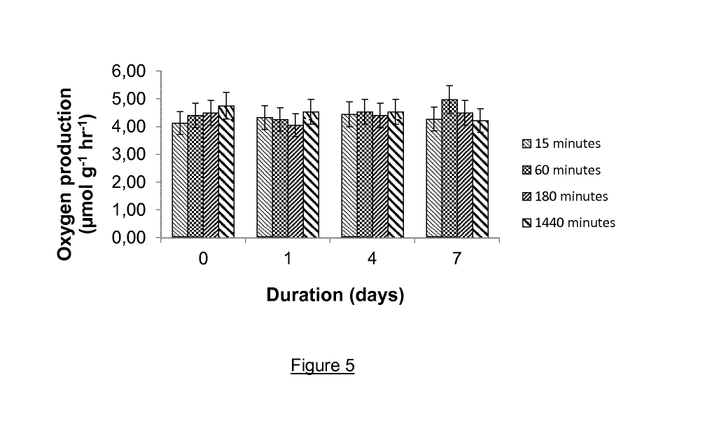

The experiment was performed as provided in example 1. The incubation time into the PDADMAC/CaCl₂ solution varied from 15 minutes to 24 hours (1440 minutes). The results are reported in FIG. 5 where the oxygen production of microalgae was analyzed at 0, 1, 4, and 7 days post-encapsulation. The incubation time of the beads in the PDADMAC/CaCl₂ solution had therefore no influence over the metabolic activity of the entrapped microalgae as shown in FIG. 5.

The invention provides the following advantages:
Hybrid alginate-silica beads of several millimeters synthesized via a one-pot, eco-friendly and low cost process exhibit a well spherical shape but also a very good mechanical and chemical stability. It is possible to adjust the size of the beads and of the pores (in the shell and in the core) by varying physical and chemical parameters of the preparation method. The obtained beads with a selected diameter and a selected pore size can be used as such in various purification methods and devices, especially in chromatographic columns.
Due to a highly porous structure throughout the entire bead volume which permits an excellent diffusion of nutrients and metabolites, large biomass of cells can be encapsulated within the beads without appearance of fracture and leakage. Thus, no growth of living cells is noted in the medium culture. Although cells can proliferate within the beads, no swelling phenomenon arises;
Regarding the maintenance of the biological activity and long-term viability of entrapped cells within the beads, cells are kept alive over at least 9 months and more.

1. Beads having a millimeter-scale size entrapping a bioactive substance, wherein the beads comprise:
a porous core, and
a porous shell, the porous core comprising a hybrid alginate-silica and the porous shell comprising silica and a silica concentrator being a long chain polyamine;
the bioactive substance being selected from the group consisting of a cell, a cell organelle, an enzyme, a drug, a pro-drug or a mixture thereof.

2. (canceled)

3. The hybrid alginate-silica beads according to claim 1, wherein the silica concentrator is the polycation PDADMAC.

4. The hybrid alginate-silica beads according to claim 1, wherein the porous shell comprises pores having a size comprised between about 1 nm and about 500 nm.

5. The hybrid alginate-silica beads according to claim 1, wherein the porous shell has a thickness comprised between about 1 μm and about 10 μm.

6. The hybrid alginate-silica beads according to claim 1, wherein an intermediate layer of hybrid calcium alginate-silica is formed between the porous core and the porous shell.

7. The hybrid alginate-silica beads according to claim 1, wherein the cells are selected from the group consisting of photosynthetically active cells, bacteria, animal cells or fungi.

8. A method for the preparation of hybrid alginate-silica beads according to claim 1, which comprises the steps of:
mixing one or more silica precursor(s) and a solution of alginate, wherein the pH of the solution is between about 2 and about 10, and with a bioactive substance to be encapsulated in said beads;
dropping the mixture into an aqueous solution of a silica concentrator comprising a long chain; and
incubating the obtained beads for a period between about 1 minute and about 24 hours.

9. (canceled)

10. The method according to claim 8, wherein the silica concentrator is the polycation PDADMAC.

11. The method according to claim 17, wherein the cationic salt is CaCl₂.

12. The method according to claim 8, wherein the silica precursor is selected from the group consisting of polysilicic acids (H₂SiO₃)ₙ, trimethoxymethylsilane, dimethoxydimethylsilane, ormosils (organic modified silicas), tetramethoxysilane (TMOS), tetraethoxysilane (TEOS), diglycerylane (DGS), and sodium silicate (N₂SiO₃) or a mixture thereof.

13. The method according to claim 8, wherein the concentration of the silica precursor in the solution comprises between about 0.1 M and about 2 M.

14. The method according to claim 8, wherein additives selected from the group consisting of silica colloids silica co-precursors or nano-particles of silica are added to the silica precursor(s) solution.

15. (canceled)

16. The hybrid alginate-silica beads according to claim 1, wherein the cells are microalgae.

17. The method according to claim 8, wherein the aqueous solution of the silica concentrator further comprises a cationic salt.
